(19) United States
(12) Patent Application Publication
(10) Pub. No.: US 2017/0065466 A1
(43) Pub. Date: Mar. 9, 2017

(54) Absorbent Core For An Absorbent Article

(71) Applicant: Principle Business Enterprises, Inc., Dunbridge, OH (US)

(72) Inventor: Andrew J. Szypka, Curtice, OH (US)

(73) Assignee: Principle Business Enterprises, Inc., Dunbridge, OH (US)

(21) Appl. No.: 15/258,264

(22) Filed: Sep. 7, 2016

Related U.S. Application Data
(60) Provisional application No. 62/215,296, filed on Sep. 8, 2015.

(51) Int. Cl. A61F 13/535; A61F 13/49; A61F 13/47

(52) U.S. Cl. CPC A61F 13/535; A61F 13/47; A61F 13/49; A61F 2013/530489

(57) ABSTRACT

The absorbent core can be utilized within absorbent article. The absorbent article has a liquid permeable nonwoven top sheet and a liquid impermeable back sheet. A cavity being formed between the back sheet and the top sheet. The absorbent core is positioned in the cavity formed between the top sheet and the back sheet The absorbent core has an acquisition layer -positioned adjacent to the top sheet and a tissue layer is positioned adjacent the acquisition layer. A plurality of SAP particles are positioned on and extend over the surface of the tissue that is spaced apart from the acquisition layer. A layer of fluff containing a multitude of super absorbent particles is positioned adjacent the tissue layer. A laminate of super absorbent particles with a layer of tissue on each side of the superabsorbent particles is positioned between the layer of Huff and the back sheet.

### CROSS REFERENCE TO RELATED APPLICATION

The present patent application is based upon and claims the benefit of provisional patent no. 62/215,298 filed Sep. 8, 2015.

### BACKGROUND OF THE INVENTION

The invention is directed to an absorbent core which can be used with an absorbent article. The absorbent article is usually an adult incontinent product, diaper, feminine hygiene product, wound care pad or absorbent pad. In the past, such absorbent articles have been capable of absorbing and retaining a quantity of bodily fluids from the user of the absorbent article, in some applications, such as overnight use of the absorbent article, the capacity of the absorbent article has been a limiting factor. The absorbent article has not had sufficient capacity to function satisfactorily for the extended time periods frequently encountered in use of the absorbent article. When the absorbent article cannot handle the quantity of fluid from the user over an extended period of time it is necessary to change the absorbent article to keep the user comfortable and sanitary. In occasions, such as overnight use, this may require the user to have interrupted sleep so that the absorbent article can be changed. If the user is In a care facility, it will also require the staff of the care facility to interact with the user during the nighttime hours. As it is difficult to determine if the capacity of the absorbent article has been exceeded without disturbing the user, this results in a disruption or interruption of the sleep cycle for the user. There is a need for an absorbent article that can handle larger quantities of fluid from the user while maintaining the user in a dry and sanitary condition. Such an enhanced absorbent article but also result in less disruption of the sleep patterns of the user.

The invention is directed to absorbent core that can be utilized within absorbent article. The absorbent article has a liquid permeable nonwoven top sheet and a liquid impermeable beck sheet. The top sheet and the back sheet have substantially the same size and shape. The top and back sheets being positioned in spaced apart aligned relationship. A cavity being formed between the back sheet and the top sheet. The absorbent core is positioned in the cavity formed between the top sheet and the back sheet. The absorbent core has an acquisition layer positioned adjacent to the top sheet and extending along the entire length and width of the cavity. A tissue layer is positioned adjacent the acquisition layer. A plurality of SAP particles are positioned on and extend over the surface of the tissue that is spaced apart from the acquisition layer. The super absorbent particles extend over the entire surface of the tissue layer. A layer of fluff containing a multitude of super absorbent particles is positioned adjacent the tissue layer. The multitude of super absorbent particles are in a layer in the layer of fluff. A laminate of super absorbent particles with a layer of tissue on each side of the superabsorbent particles is positioned between the layer of fluff and the back sheet.

Other objects and advantages of the present invention will become apparent to those skilled in the art upon a review of the following detailed description of the preferred embodiments and the accompanying drawings.

### BRIEF DESCRIPTION OF THE DRAWINGS

FIG. 1 is a cross-sectional side elevational view of an absorbent article utilizing the absorbent core of the present invention.

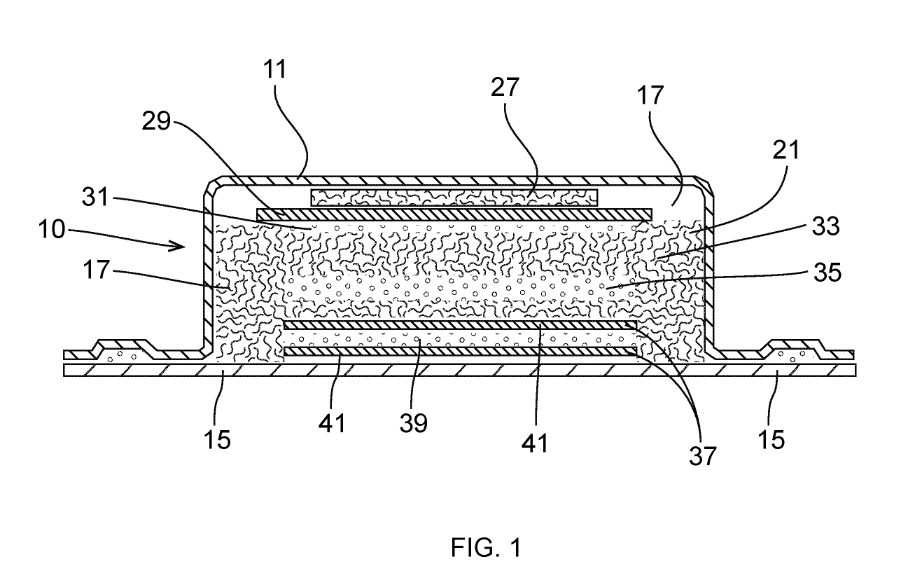

FIG. 2 is a plan view of an absorbent article with a partially exposed cross-sectional portion showing the absorbent core of the present invention.

### DETAILED DESCRIPTION OF THE PREFERRED EMBODIMENTS(S)

The present invention is directed to an absorbent core that is utilized in an absorbent article. More particularly, the absorbent core is designed to be positioned in the absorbent article in a location that maximizes the retention of fluids directed to the absorbent article. The features of the invention will be more readily understood by referring to the attached drawings in connection with the following description.

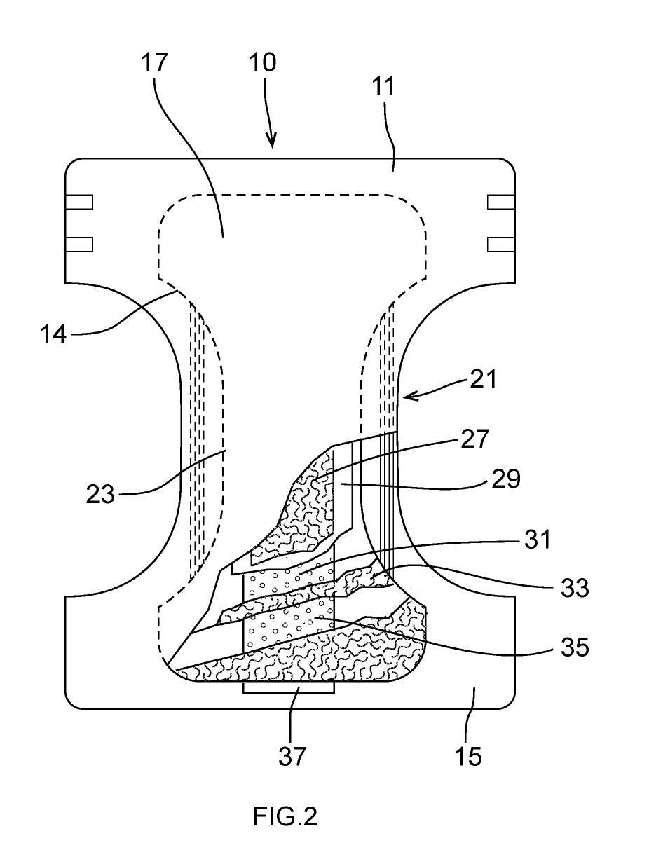

The absorbent article has a top sheet 11 that is formed of a fluid permeable material and a back sheet that is formed of a fluid impervious plastic or laminated film. The top sheet 11 is joined to the back sheet 15 around the outer periphery of the two sheets in a manner well-known in absorbent articles. The top sheet 11 is positioned to be adjacent the user of the absorbent article and the back sheet is positioned to be spaced apart from the user of the absorbent article. A portion of the top sheet 11 is spaced apart from the back sheet to form a cavity 17. An absorbent, core 21 is positioned in the cavity 17. The absorbent core is designed to distribute and hold fluids from the user that passed through the top sheet 11. FIG. 2 shows one example of the absorbent article that can use the absorbent core of the present invention. The absorbent article of FIG. 2 is an incontinent device, such as, a diaper. The absorbent article has a length (L) and a width (W) with a reduced width section 23 located in the central region of the absorbent article. The reduced width section 23 has a width that is from about 30% to about 80% of the width W for the absorbent article. The absorbent article has a generally hoar glass shape, but it should be appreciated that other shapes can be used for the absorbent article. The cavity 17 that contains the absorbent core 21 is shown in dashed lines 14 in FIG. 2. The cavity 17 has a shape that generally conforms to the shape of the absorbent article formed by the top sheet 11 and the back sheet 15.

The absorbent core 21 has several components that are utilized to distribute and hold fluids from the user of the absorbent article. Positioned adjacent the top sheet 11 is an acquisition layer 27. The acquisition layer is a polyester non-woven material that extends the entire length of the cavity and from about 48% to about 58% of the width of the reduced width section 23 of the cavity 17. The acquisition layer 27 is designed to quickly distribute fluids that pass through the top sheet 11. Positioned adjacent the side of acquisition layer that is spaced apart from the top sheet 11 is a layer of tissue 29 having a plurality of super absorbent particles 31 bonded to the layer of tissue. The layer of tissue with super absorbent particles extends from about 85% to about 95% of the width of the reduced width section 23 the width and the entire length of the cavity 17 The layer of tissue 29 has a weight from about 8 to about 18 pounds per 3000 square ft. The plurality of super absorbent particles 31 are bonded to the tissue by water spray and then pressure and optionally heat is used to secure the super absorbent particles to the tissue layer. The plurality of super absorbent particles 31 are round particles having an absorption capacity from about 50 g/g to about 65 g/g The super absorbent particles are from about 80% to about 90% by weight of the layer of tissue 29 and super absorbent particles 31 The super absorbent particles 31 have a gel strength (AUL) from about 18 g/g to about 24 g/g.

Positioned next in the absorbent core 21 is a layer of fluff 33 containing a layer of super absorbent particles 35. The layer of super absorbent particles are positioned in a discrete layer in the layer of fluff during the manufacture of the fluff. The plurality of super absorbent particles 31 secured to the layer of tissue 29 are in contact with the surface of the layer of fluff that is spaced apart from the layer of super absorbent particles 35. The layer of fluff is formed of fibers of cellulose that have a density from about 0.1 g/cu.cm to about 0.29/cu.cm The layer of super absorbent particles 35 are round particles having an absorption capacity from about 50 g/g to about 65 g/g. The super absorbent particles 35 have a gel strength from about 12/g to about 24/g and from about 4% to about 12% by weight of the layer of fluff and super absorbent particles. The layer of fluff 33 is positioned centrally in the cavity 17 and extends substantially the entire width and length of the cavity. The layer of super absorbent particles 35 are in a layer that has a length that is from about ½ to about ¾ of the length of the cavity. The layer of super absorbent particles are usually positioned in the center of the layer of fluff, but other positions can be used to modify the absorption properties of the layer of fluff. The fibers of the fluff hold the super absorbent particles 35 in the desired position. Usually, the plurality of super absorbent particles is positioned within a zone that is spaced from about 25% to about 75% from the surface of the layer of fluff 33 that is adjacent the layer of tissue 29, The layer of super absorbent particles 35 has a width that is from about 75% to about 85% of the width of the reduced width section 23 of the cavity.

In practice it has been found that the plurality of super absorbent particles 31 can be positioned on the surface of the layer of fluff 33 that is adjacent the layer of tissue 29. Water is then sprayed onto the surface of the fluff and the plurality of super absorbent particles. The layer of tissue is then positioned over the surface of the fluff. Pressure and optionally heat is then applied to the layer of tissue 29 and the layer of fluff 33 to secure the plurality of super absorbent particles 31 between the layers of tissue and fluff. The plurality of super absorbent particles 31 are secured by hydrogen bonding that is created by the application of pressure to the moist surfaces containing the plurality of super absorbent particles. The pressure can be applied by an embossing process.

Positioned next in the absorbent core is a laminate 37 of a multitude of super absorbent particles 39 with a layer of tissue 41 positioned on each side of the super absorbent particles. The laminate is two layers of tissue 41 with the multitude of super absorbent particles positioned between the layers of tissue. The laminate 37 is positioned between the layer of fluff 33 and the back sheet 15. The multitude of super absorbent particles 39 in the laminate 37 have the same shape and absorption parameters as the super absorbent particles 35 positioned in the layer of fluff. Tissue used in the laminate 37 has a weight from about 10 lbs. to about 20 lbs. per 3,000 square feet. The laminate 37 has the same length as layer of super absorbent particules 35 that are in the layer of fluff and the laminate is also centrally positioned in the cavity 17. The laminate has a width that is from about 50% to about 85% of the width of the reduced width section 23 of the cavity 17 which is slightly narrower than the layer of fluff 33. The laminate is an ultra-thin, cellulosic, ultra-dense structure. The laminate has from about 60% to about 80% of SAP particles by weight with a thickness of less than 25 mils.

The portion of the absorbent core 21 from the tissue layer 29 down to the laminate 37 can be embossed to assist in maintaining these components in the desired position in the absorbent article 10.

In operation, the acquisition layer 27 acts to draw fluid through the pervious top sheet 11 info the absorbent core 21. The acquisition layer also acts to wick the fluid from the user along the acquisition layer to distribute the fluid along the absorbent core. The plurality of SAP particles 31 in the tissue layer 29 are designed to have a slower absorption rate than the SAP particles 35, 39 in the layer of fluff 33 and the laminate 37. Accordingly, the plurality of SAP particles 31 in the tissue layer 29 do not absorb ail of the fluid from the user, but allows the fluid to pass to the layer of fluff 33. The layer of SAP particles 35 in the layer of fluff absorb more of the fluid in a quicker manner than the plurality of SAP particles 31 in the layer of tissue. The fibers in the fluff that surrounds the SAP particles 35 act to distribute the fluid throughout the fluff and to more SAP particles that are located in the fluff. The layer of fluff 33 and layer of SAP particles 35 do a very good job of distributing and absorbing the fluid from the user. If there is more fluid than the layer of fluff can handle, the fluid moves through the layer of fluff to the laminate 37. The multitude of SAP particles 39 in the laminate have the same basic characteristics as the layer SAP particles 35 in the layer of Huff and act to quickly absorb any fluids that come into contact with the laminate 37. The layers of tissue 41 that are part of the laminate also function to distribute the fluid along the laminate to allow the multitude of SAP particles 39 to absorb any excess field that is not absorbed in the layer of fluff 33 or the layer of tissue 29-SAP particle 31 layer.

As the plurality of SAP particles 31 adjacent the acquisition layer absorb more slowly, the majority of the absorption and retention of the fluid from the user is accomplished by the layer of fluff 33 and the laminate 37. This results in the fluid being retained in layers of the absorbent core 21 that are spaced apart from the user which results in the user being kept in a dry condition. As the fluid is retained primarily in the SAP particles of the various layers, there is a reduced incidence of rewetting as the fluid cannot move back through the absorbent core 21 and come into contact with the user.

The laminate layer 37 and layer of SAP particles 35 in the layer of fluff 33 are positioned in the absorbent article where the largest volume of fluid is received from the user. This allows these layers to function effectively without adding unnecessary material to the absorbent article. The SAP particles in various layers of the absorbent core 21 are held in place by layers of tissue or by the layer of fluff. Accordingly, there are no issues of loose SAP particles moving to undesirable locations in the absorbent article. The tissue layers and the layer of fluff also act as buffer layers that keep the SAP particles from coming into direct contact with the top sheet or the back sheet. The buffer layers eliminate the occurrence of pin holes that can be created when the SAP particles come into contact with the top or back sheet when pressure is applied to the absorbent article. The different densities of the layers of the absorbent core 21 manage the distribution and retention of the fluids from the user in a way that the user is kept dry with little or no occurrences of rewetting when pressure is applied to the absorbent article 10. The laminate layer 37 is an ultra-dense, ultra-thin, non-pulp structure that can absorb and retain additional quantities of fluid while allowing the absorbent core 21 to remain sufficiently thin to be comfortable for the user. The thinness of the core also reduces freight cost and the need for additional shelf space for this higher performing absorbent product.

The above detailed description of the present invention is given for explanatory purposes. It will be apparent to those skilled in the art that numerous changes and modifications can ha made without departing from the scope of the invention. Accordingly, the whole of the foregoing, description is to be construed in an illustrative and not a limitative sense, the scope of the invention being defined solely by the appended claims.

I claim,

1. An absorbent core for an absorbent article having a liquid permeable nonwoven fop sheet and a liquid impermeable back sheet, the top sheet and back sheet having substantially the same size and shape, the top and back sheets being positioned in spaced apart aligned relationship, a cavity being formed between the top sheet and the back sheet, the absorbent core comprising;
an acquisition layer positioned in the cavity adjacent the top sheet and extending along the entire length and width of the cavity;
a tissue layer positioned adjacent the acquisition layer;
a plurality of SAP particles positioned on and extending over the entire surface of the tissue layer that is spaced apart from the acquisition layer;
a layer of fluff is positioned adjacent the tissue layer;
a layer of SAP particles positioned in the interior of the layer of fluff;
a laminate having two layers of tissue with a multitude of SAP particles positioned between the two layers of tissue, the laminate being positioned between the layer of fluff and the back sheet.

2. The article of claim 1 wherein the layer of fluff containing SAP particles is centered on the top sheet and extends along the length of the top sheet.

3. The article of claim 2 wherein the layer of SAP particles extends from about ½ to about ¾ of the length of the top sheet.

4. The article of claim 2 wherein the laminate has the same length and position with respect to the top sheet as the layer of SAP particles in the layer of fluff.

5. The article of claim 3 wherein the laminate of a multitude of SAP particles and tissue has a width that is from about 50% to about 85% of the width of the layer of fluff.

6. The article of claim 1 wherein the plurality of SAP particles have an absorption capacity from about 50 g/g to about 85 g/g.

7. The article of claim 1 wherein the top and back sheets have an hour glass shape with a reduced width section in the center and a wider section positioned on each side of the reduced width section.

8. The article of claim 7 wherein the reduced width section has a width that is from about 30% to 60% of the width of the wider section.

9. The article of claim 8 wherein, the layer of tissue and SAP articles extends from about 85% to about 95% of the width of the reduced width section.

10. The article of claim 8 wherein the layer of SAP particles has a width that is from about 75% to about 85% of the width of the reduced width section.
